(19) United States
(12) Patent Application Publication
(10) Pub. No.: US 2011/0098563 A1
(43) Pub. Date: Apr. 28, 2011

(54) ULTRASONIC DIAGNOSTIC APPARATUS, ULTRASONIC IMAGE DISPLAY METHOD AND ULTRASONIC DIAGNOSTIC PROGRAM

(76) Inventor: Takashi Osaka, Tokyo (JP)

(21) Appl. No.: 12/999,401

(22) PCT Filed: Jun. 11, 2009
(86) PCT No.: PCT/JP2009/060688

§ 371 Date: Dec. 16, 2010

(30) Foreign Application Priority Data
Jun. 16, 2008 (JP) 2008-156076

(51) Int. Cl. A61B 8/00

(52) U.S. Cl. 600/438

(57) ABSTRACT

An ultrasonic diagnostic apparatus, an ultrasonic image display method and an ultrasonic diagnostic program by which a display mode of a tomographic image or an elasticity image can be set according to the characteristics of a biological tissue are provided. The ultrasonic diagnostic apparatus is characterized in comprising an elasticity information analyzing unit which sets a plurality of regions of interest on a tomographic image or an elasticity image and analyzes the feature amount of the elasticity information in the plurality of regions of interest; and a hue setting unit which sets the hue of the elasticity image on the basis of the feature amount.

### FIELD OF THE INVENTION

The present invention relates to an ultrasonic diagnostic apparatus, ultrasonic image display method and ultrasonic diagnostic program for displaying a tomographic image and an elasticity image that indicates softness or hardness of biological tissues in an examination target area of an object to be examined using ultrasonic waves.

### DESCRIPTION OF RELATED ART

An ultrasonic diagnostic apparatus transmits ultrasonic waves to the inside of an object to be examined by an ultrasonic probe, constructs, for example a tomographic image based on the reception signals received from biological tissues inside of the object, and displays the constructed image.

Also, it measures the reception signals received from biological tissues in the object by an ultrasonic probe, and obtains displacement in the respective areas of the body from the two sets of RF signal frame data of the reception signals measured at different times. Then an elasticity image that indicates strain or elasticity modulus of biological tissues is generated based on the obtained displacement data (for example, Patent Document 1). Further, an elasticity image that indicates strain or elasticity modulus is generated using pulsation which is autonomic biological motion (For example, Patent Document 2).

### CONVENTIONAL ARTS

Patent Document 1: JP-A-2004-135929
Patent Document 2: WO2006/132203

However, in the case of diagnosing plaque of biological tissues (particularly carotid artery) using an ultrasonic diagnostic apparatus, it is highly possible that detailed tissue characterization of plaque is overlooked in a screening examination, etc. since plaque is a comparatively small section. Also, hardness of biological tissues is not always different in the case such as when the tissue itself constructing plaque is the same tissue as plaque such as plaque filled with fibrous tissues.

The objective of the present invention is to provide an ultrasonic diagnostic apparatus, ultrasonic image display method and ultrasonic diagnostic program capable of setting display mode of a tomographic image or elasticity image in accordance with the characteristics of biological tissues.

### BRIEF SUMMARY OF THE INVENTION

In order to achieve the above-mentioned objective, the ultrasonic diagnostic apparatus of the present invention comprises:

an ultrasonic probe configured to transmit/receive ultrasonic waves to/from an object to be examined;

a transmission unit configured to transmit ultrasonic waves via the ultrasonic probe;

a reception unit configured to receive the reflected echo signals from the object;

an elasticity information calculating unit configured to calculate strain or elasticity modulus by the RF signal frame data based on the reflected echo signal received by the reception unit;

an elasticity image constructing unit configured to construct an elasticity image based on the strain or elasticity modulus acquired by the elasticity information calculation unit;

a tomographic image constructing unit configured to construct a tomographic image based on the RF signal frame data; and

an image display unit configured to display the tomographic image or the elasticity image,

characterized in further comprising:

an elasticity information analyzing unit configured to set a plurality of regions of interest on the tomographic image or the elasticity image, and to analyze feature amount of the elasticity information in the set plurality of regions of interest; and

a hue setting unit configured to set a hue of the elasticity image based on the feature amount.

Also, the ultrasonic image display method in the present invention has:

a step that constructs an elasticity image based on strain or elasticity modulus by ultrasonic signals;

a step that constructs a tomographic image by ultrasonic signals;

a step that sets a plurality of regions of interest on the tomographic image or the elasticity image;

a step that analyzes feature amount of the elasticity information on the plurality of regions of interest;

a step that sets a hue of the elasticity image based on the feature amount; and

a step that displays the elasticity image based on the set hue.

In accordance with the present invention, it is possible to set a display mode of a tomographic image or elasticity image according to the characteristics of biological tissues. Therefore, the hue in the same tissue can be clarified, so a diseased area due to the same tissue can be easily identified.

In this manner, in the present invention, it is possible to set a display mode of a tomographic image or elasticity image in accordance with the characteristics of biological tissues.

### BRIEF DESCRIPTION OF THE DIAGRAMS

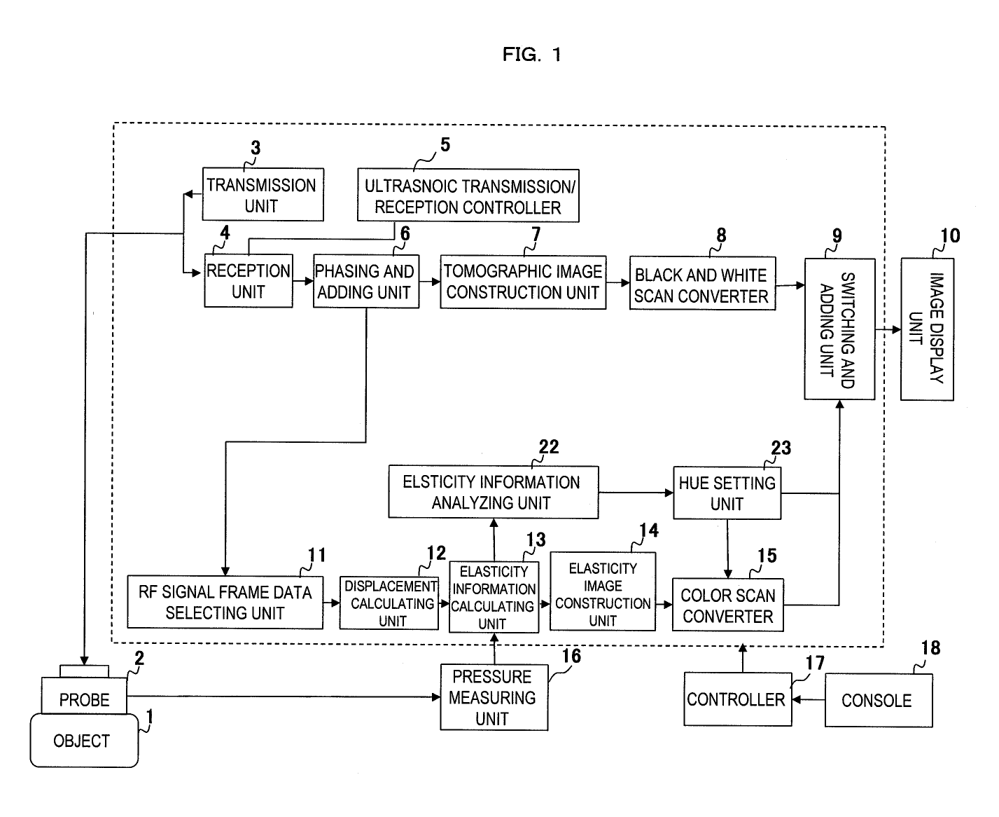

FIG. 1 shows device configuration of a first, fourth and fifth embodiments related to the present invention.

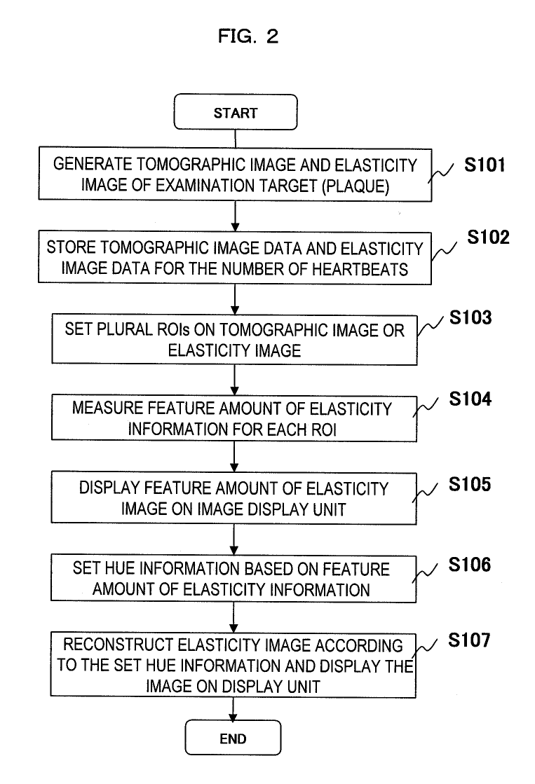

FIG. 2 shows operation procedure of the first embodiment and a second embodiment related to the present invention.

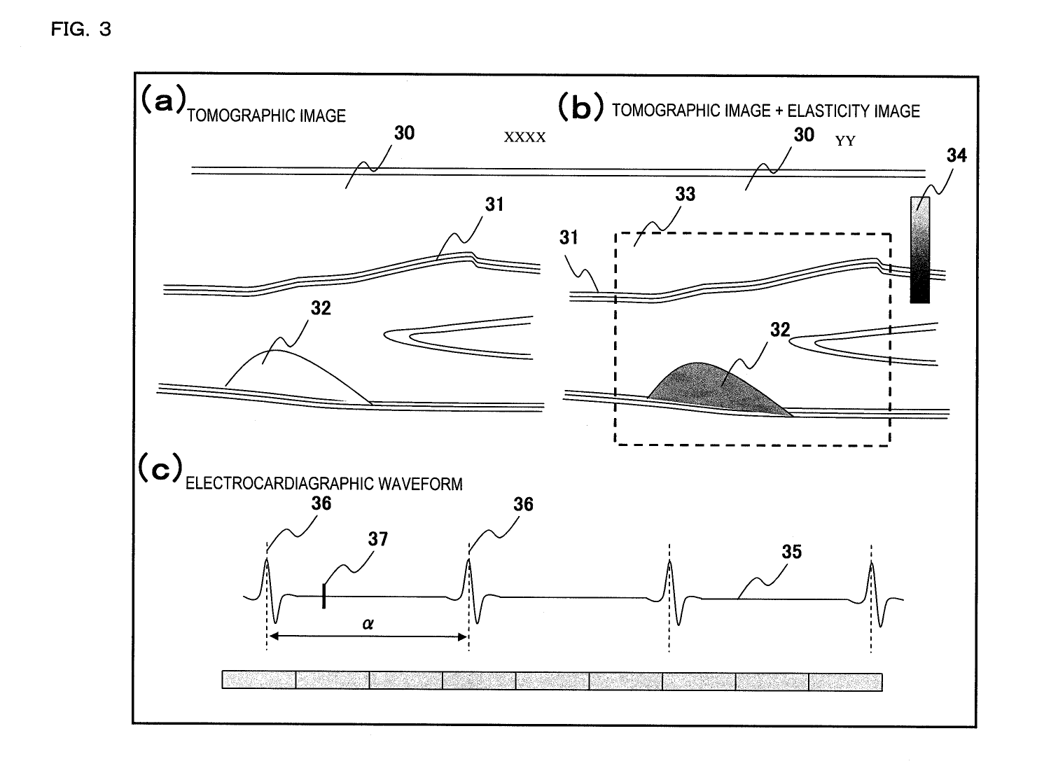

FIG. 3 shows a display mode related to the present invention.

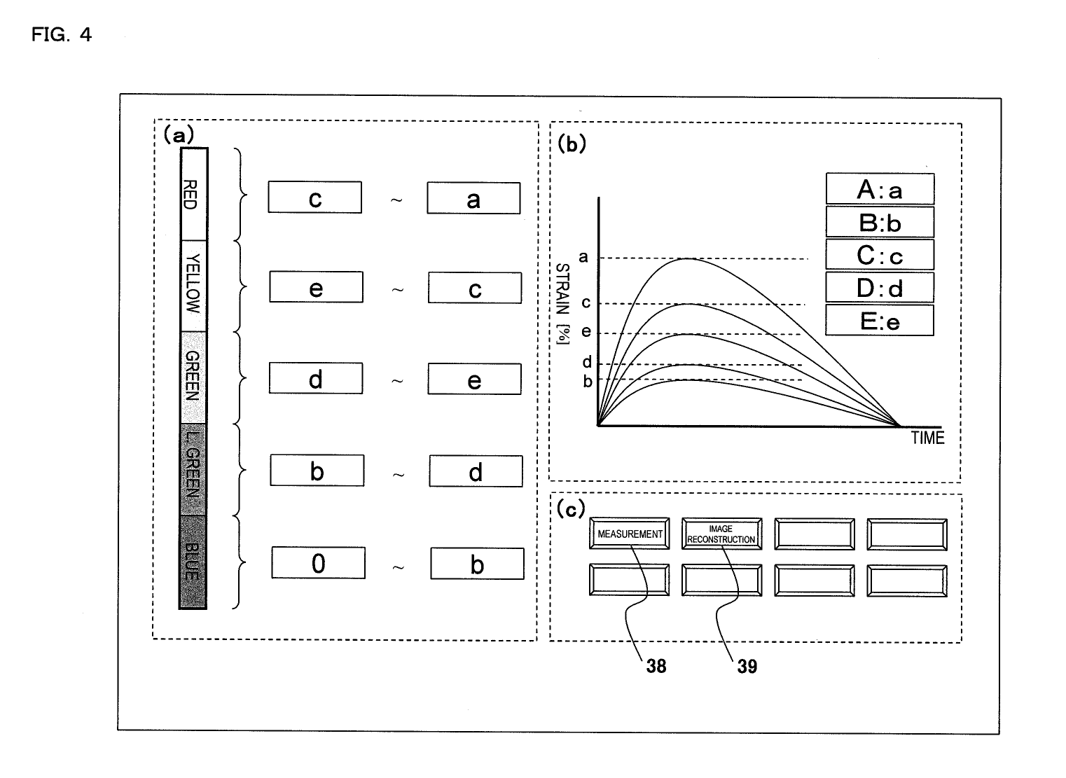

FIG. 4 shows elasticity information analysis, hue setting, etc. related to the present invention.

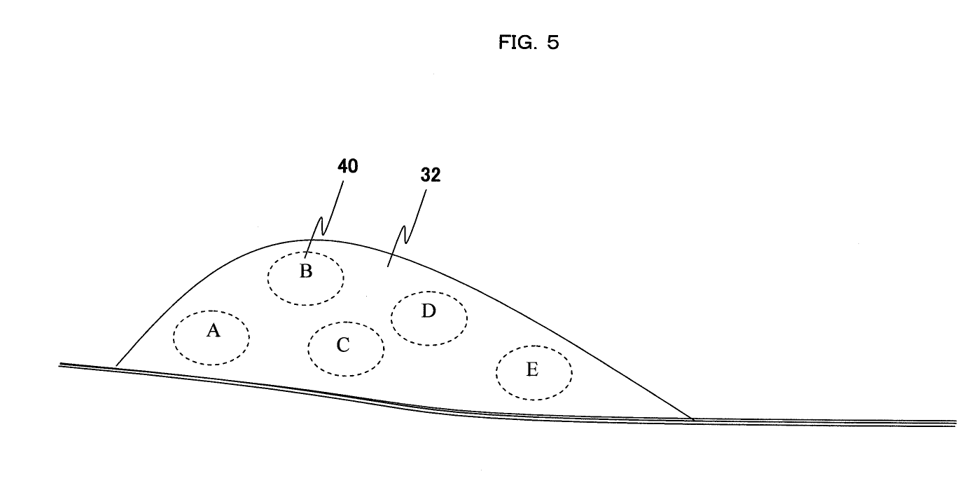

FIG. 5 shows the setting of a plurality of regions of interest related to the present invention.

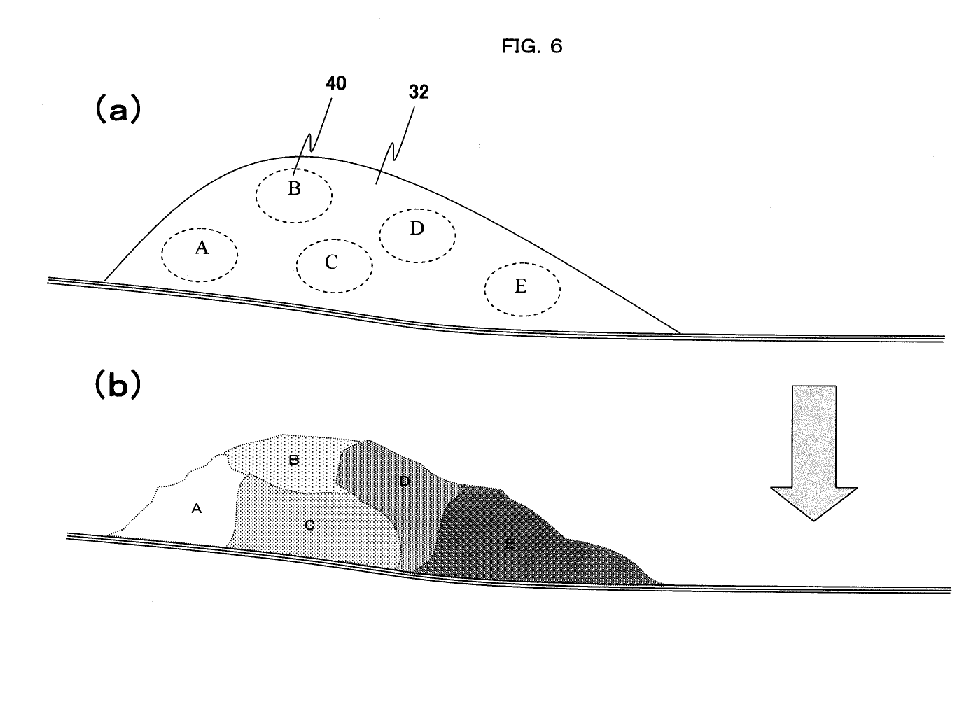

FIG. 6 shows elasticity images before and after being reconstructed.

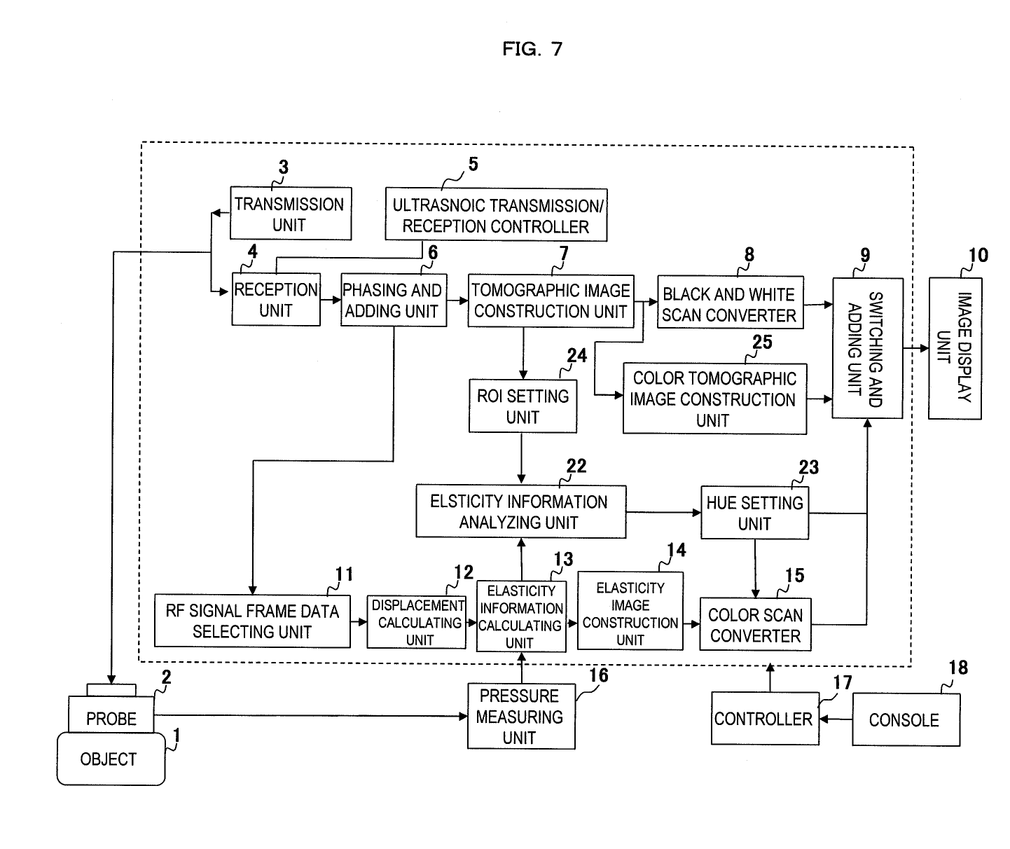

FIG. 7 shows a device configuration of the second˜fifth embodiments.

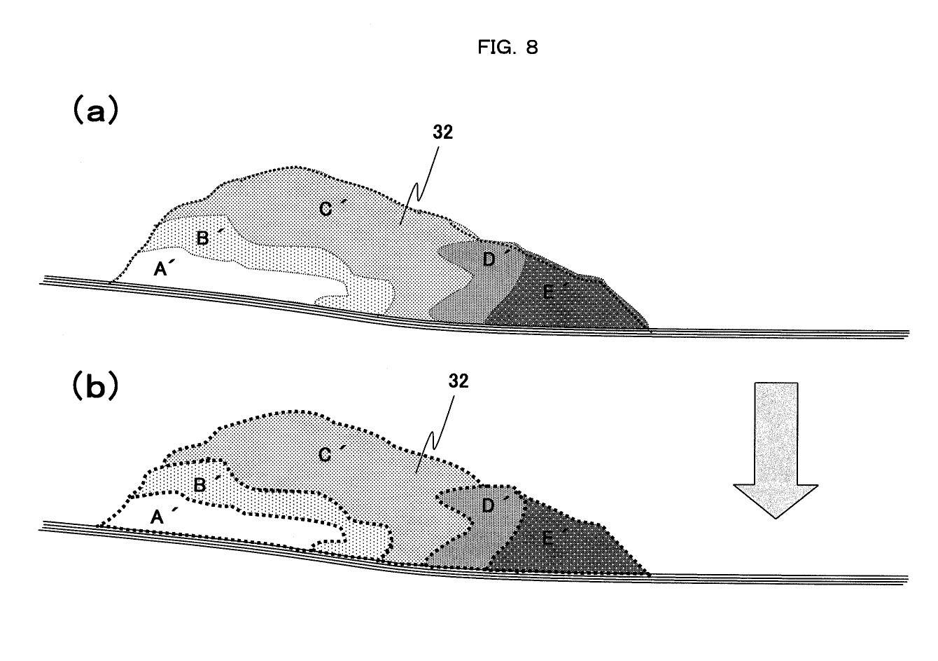

FIG. 8 shows details of the second embodiment related to the present invention.

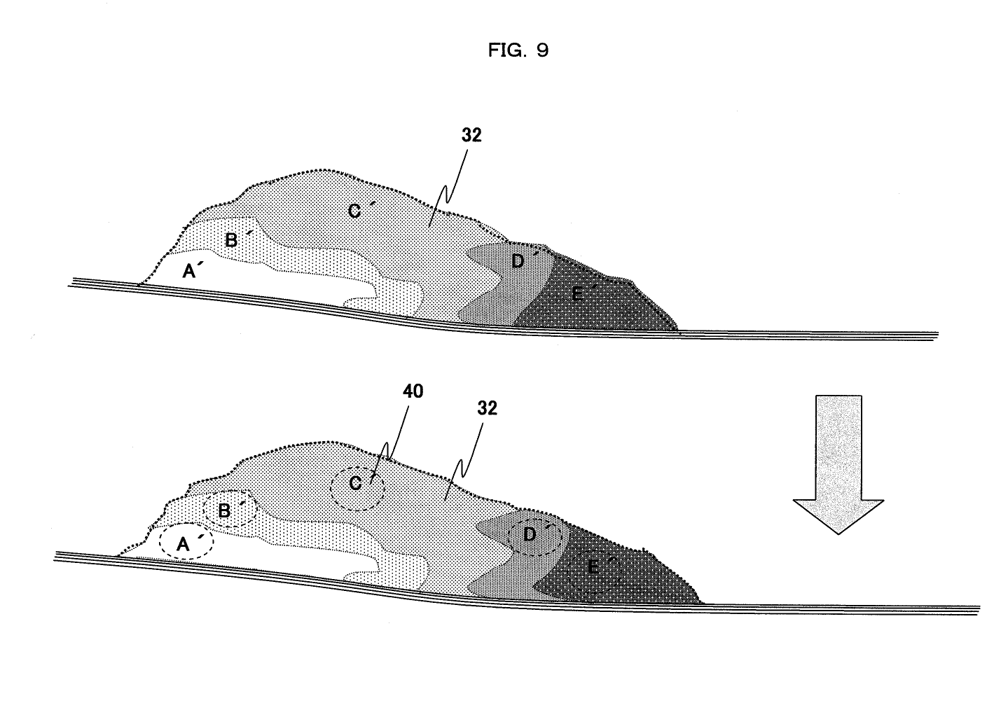

FIG. 9 shows re-setting of a region of interest related to the present invention.

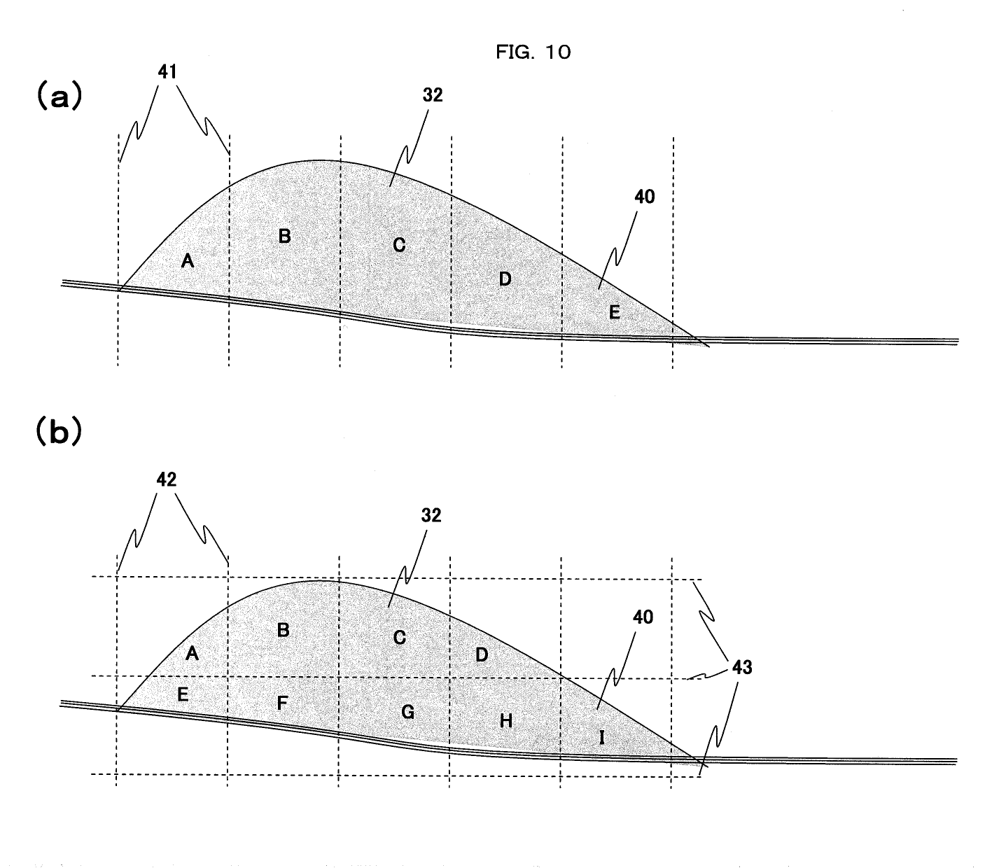

FIG. 10 shows details of the second embodiment related to the present invention.

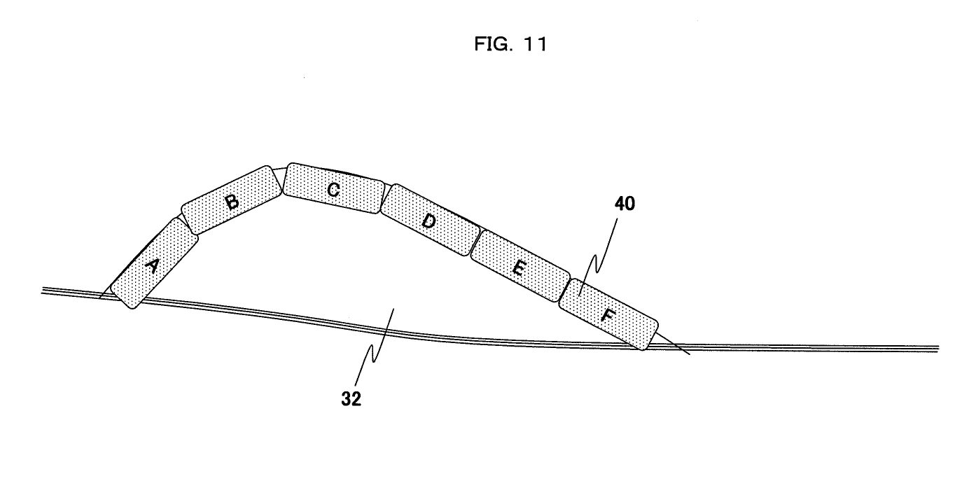

FIG. 11 shows details of the second embodiment related to the present invention.

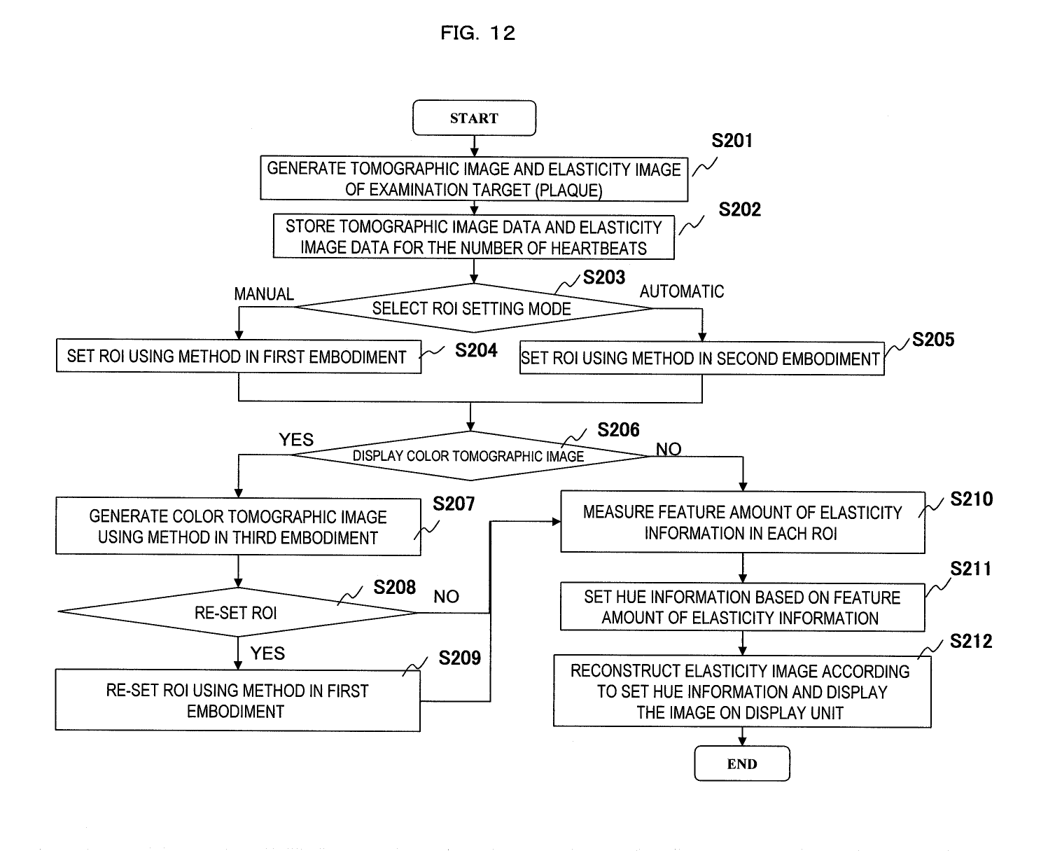

FIG. 12 shows operation procedure in the case that the first˜fifth embodiments related to the present invention are combined.

### DETAILED DESCRIPTION OF THE INVENTION

### First Embodiment

### Manual

The ultrasonic diagnostic apparatus to which the present invention is applied will be described referring to FIG. 1. As shown in FIG. 1, the ultrasonic diagnostic apparatus comprises ultrasonic probe 2 to be used by applying to object 1, transmission unit 3 configured to repeatedly transmit ultrasonic waves to object 1 via ultrasonic probe 2 spacing time intervals, reception unit 4 configured to receive the time-series reflected echo signals produced from object 1, ultrasonic transmission/reception controller 5 configured to switch the transmission and the reception of transmission unit 3 and reception unit 4, and phasing and adding unit 6 configured to perform phasing and adding of the reflected echo signals received by reception unit 4.

The ultrasonic diagnostic apparatus also comprises tomographic image constructing unit 7 configured to construct a grayscale tomographic image, for example a black and white tomographic image of object 1 based on the RF signal frame data from phasing and adding unit 6, and black and white scan converter 8 configured to convert the output signals from tomographic image constructing unit 7 into the signals suited for display on image display unit 10.

The ultrasonic diagnostic apparatus further comprises RF signal frame data selecting unit 11 configured to store the RF signal frame data outputted from phasing and adding unit 6 and to select at least two sets of frame data, displacement calculating unit 12 configured to calculated displacement of biological tissues in object 1, elasticity information calculating unit 13 configured to acquire elasticity information such as strain or elasticity modulus from the displacement information measured in displacement calculating unit 13, elasticity image constructing unit 14 configured to construct a color elasticity image from the strain or elasticity modulus calculated in elasticity information calculating unit 13, color scan converter 15 configured to convert the output signals from elasticity image construction unit 14 into the signals suited for displaying on image display unit 10, switching and adding unit 9 configured to switch the display modes by superimposing a black and white tomographic image and an elasticity image or juxtaposing the images, and image display unit 10 configured to display a tomographic image, and an image in which a tomographic image and an elasticity image are synthesized.

In the case that elasticity modulus is calculated in elasticity information calculating unit 13, the pressure information acquired by pressure measuring unit 16 connected to a pressure sensor (not shown in the diagram) of ultrasonic probe 2 is outputted to elasticity information calculating unit 13.

Also, the ultrasonic diagnostic apparatus comprises controller 17 configured to control the respective components and console 18 configured to input various information to controller 17. Console 18 comprises a device such as a keyboard or trackball.

Here, the respective components will be described in detail. Ultrasonic probe 2 is formed by a plurality of transducers disposed therein, for transmitting and receiving ultrasonic waves to/from object 1 via the transducers. Transmission unit 3 generates transmission pulses for producing ultrasonic waves by driving ultrasonic probe 2, and sets the convergent point of the transmitted ultrasonic waves at a certain depth. Reception unit 4 amplifies the reflected echo signals received by ultrasonic probe 2 at a predetermined gain, and generates RF signals, i.e. reception signals. Ultrasonic transmission/reception controller 5 controls transmission unit 3 and reception unit 4.

Phasing and adding unit 6 inputs the RF signals amplified in reception unit 4 to execute phase control, and generates RF signal frame data by forming an ultrasonic beam with respect to one or plural convergent points.

Tomographic image constructing unit 7 inputs the RF signal frame data from phasing and adding unit 6 and executes signal processes such as gain compensation, log compression, detection, edge enhancement or filtering, so as to obtain tomographic image data. Also, black and white scan converter 8 is provided with an A/D converter for converting the tomographic image data from tomographic image constructing unit 7 into digital signals. Black and white scan converter 8 acquires tomographic image data as one image, and reads out the acquired tomographic image data with TV synchronism.

RF signal frame data selecting unit 11 stores the plural sets of RF signal frame data from phasing and adding unit 6, and selects two sets, i.e. a pair of RF signal frame data from among the stored RF signal frame data group.

RF signal frame data selecting unit 11 sequentially stores the RF signal frame data generated by phasing and adding unit 6 in time series and selects the stored RF signal frame data (N) as a first data, at the same time as selecting one RF signal frame data (X) from among RF signal frame data group (N−1, N−2, N−3, . . . N−M) that are temporally stored in the past. Here, N, M and X are index numbers appended to RF signal frame data, and are positive integers.

Then displacement calculating unit 12 acquires displacement or a moving vector of the biological tissues corresponding to the respective points on a tomographic image, i.e. one-dimensional or two-dimensional displacement distribution related to the direction and size of the displacement, by executing one-dimensional or two-dimensional correlation process from a selected pair of data, i.e. RF signal frame data (N) and RF signal frame data (X). Here, the block matching method is used for detecting moving vectors. The block matching method divides an image into blocks formed by, for example N×N pixels, focuses on the block in a region of interest, searches the block which is the most approximated to the focused block from the previous frame, and determines the sample value by predictive coding, i.e. difference referring to the searched block.

Elasticity information calculating unit 13 calculates strain or elasticity modulus of the biological tissues corresponding to the respective points on a tomographic image from the measured value, for example the moving vector outputted from displacement calculating unit 12 and the pressure value outputted from pressure measuring unit 16, and generates elasticity information.

At this time, strain is calculated by performing spatial differentiation on the moving amount, for example the displacement of biological tissues. Also, elasticity modulus is calculated by dividing the variation of pressure by the variation of strain. For example, by setting the displacement measured by displacement calculating unit 12 as L(X) and the pressure measured by pressure measuring unit 16 as P (X), since strain SΔ(X) can be calculated by performing spatial differentiation on L(X), it can be obtained using the equation which is ΔS(X)=ΔL(X)/ΔX. Also, Young's modulus Ym(X) of elasticity modulus can be calculated by the equation which is Ym=(ΔP(X))/ΔS(X). Since elasticity modulus of the biological tissues equivalent to the respective points on the tomographic image can be obtained from this Young's modulus Ym, two-dimensional elasticity images can be continuously obtained. Young's modulus is the ratio with respect to the simple tensile stress added to a subject and the strain generated parallel to tension.

Elasticity image constructing unit 14 executes various image processing such as the smoothing process in a coordinate plane, contrast optimization process or smoothing process in the time-axis direction between frames with respect to the calculated elasticity information (strain and elasticity modulus), and constructs elasticity image data.

Color scan converter 15 has a function to append a hue to the elasticity image data outputted from elasticity image constructing unit 14. More specifically, it converts the image data into light's three primary colors, i.e. red(R), green(G) and blue(B). For example, it converts the elasticity data having large strain into a red color code, and the elasticity data having small strain into a blue color code.

Switching and adding unit 9 is configured formed by a frame memory, an image processing unit and an image selecting unit. Here, the frame memory stores the data such as the tomographic image data outputted from black and white scan converter 8 and the elasticity image data outputted from color scan converter 15 along with time information. Also, the image processing unit synthesizes the tomographic image data and elasticity image data stored in the frame memory by changing the synthesis ratio. Luminance information and hue information of the respective pixels in a synthesized image is added by the synthesis ratio for each coordinate. Further, the image selecting unit selects the image to be displayed on image display unit 10 from among the tomographic image data and the elasticity image data in the frame memory and the synthesized image data in the image processing unit.

Further, the ultrasonic diagnostic apparatus in the present embodiment comprises elasticity information analyzing unit 22 configured to read out elasticity information at a predetermined time in a plurality of regions of interest being set on a tomographic image or an elasticity image from elasticity information calculating unit 13 and analyzes elasticity information at that time, and hue setting unit 23 configured to set a hue of elasticity image data based on the feature amount in the respective regions of interest analyzed by elasticity information analyzing unit 22. They will be described in concrete terms referring to FIG. 2-FIG. 6. FIG. 2 is operation procedure of the first embodiment (includes the ultrasonic image display method and ultrasonic diagnostic program).

(S101)

First, Tomographic Image Constructing Unit 7 and elasticity image constructing unit 14 construct tomographic image data and elasticity image data of biological tissues (plaque 32 of carotid artery 30 in the present embodiment) and display the constructed data on image display unit 10. FIG. 3 shows image display modes of image display unit 10. FIG. 3(a) shows the display of a tomographic image, and FIG. 3(b) shows the image in which an elasticity image is overlapped with a tomographic image. The tomographic image and the elasticity image are displayed on the same screen, and the phases of the displayed tomographic image and the elasticity image are matched.

As shown in FIGS. 3(a) and (b), plaque 32 which is a biological tissue is displayed in carotid artery 30. Plaque 32 is attached to wall 31 of carotid artery 30 by cholesterol, etc. and is a factor of arterial sclerosis.

Also, region frame 33 shown in FIG. 3(b) indicates the calculation/display region of an elasticity image. The size or pattern of region frame 33 can be set as desired by console 18. The reason for setting region frame 33 is to reduce calculations to be performed by displacement calculating unit 12 and elasticity information calculating unit 13, etc. for displaying an elasticity image. Wall 31 of carotid artery 30 or plaque 32 within region frame 33 is colored to be displayed.

Scale 34 is for corresponding strain or elasticity modulus which is elasticity information to a hue in elasticity image. The hue information of scale 34 set by console 18 is transmitted to the hue information of color scan converter 15 by controller 17. By adjusting hue information of scale 34 by console 18, the hue can be set by converting the elasticity image data having large strain into a red color code or converting the elasticity image data having small strain into a blue color code.

FIG. 3(c) is electrocardiogram 35 of an object. Though not shown in the diagram, an electrode is attached to object 1 for measuring electrocardiogram 35. Details of the method for measuring electrocardiogram 35 will be omitted since it is a commonly known technique. The electrical signals outputted from the electrode attached to object 1 are to be corresponded to tomographic image data and elasticity image data, and stored in an electrocardiogram memory (now shown in the diagram) in switching and adding unit 9 as electrocardiogram data.

In the frame memory in switching and adding unit 9, plural sets of tomographic image data and elasticity image data for one or more cycles of heartbeats are stored being corresponded to the electrocardiographic data. In the present embodiment, it is assumed that tomographic image data and elasticity image data for 3 cycles of heartbeats are stored in the frame memory. Image display unit 10 reads out the electrocardiographic data from the electrocardiogram memory along with the tomographic image data and elasticity image data, and displays electrocardiogram 35 along with a tomographic image and an elasticity image.

The α-period of electrocardiogram 35 shown in FIG. 3(c) is a heartbeat period for one cycle which is between two R-wave time phases 36. Electrocardiographic bar 37 shows the time phase when the tomographic image and the elasticity image to be displayed on image display unit 10 are acquired. The display time phase of the tomographic image or the elasticity image can be specified by operating the position of electrocardiagraphic bar 37 to the left or the right by console 18. Then controller 17 reads out the tomographic image data and elasticity image data at the time phase thereof from the frame memory, and causes image display unit 10 to display electrocardiogram 35 along with the tomographic image and the elasticity image.

(S102)

The Tomographic Image and the Elasticity Image are to be frozen using console 18. Elasticity information calculating unit 13 stores the calculated elasticity information for a several heartbeat periods before the time phase was frozen. When the tomographic image and the elasticity image are frozen by console 18, the screen shown in FIG. 4 is substituted with electrocardiograph 35 shown in FIG. 3(c) and displayed on image display unit 10.

(S103)

As shown in FIG. 5, the Operator Sets a Plurality of Regions of interest 40 in the biological tissue on the frozen tomographic image or elasticity image using console 18. Via console 18, the setting of position, shape, size, numbers, etc. of regions of interest 40 is to be executed. Here, regions of interest A˜E are set in plaque 32 which is a biological tissue.

While region of interest 40 is set using console 18 in the present embodiment, it may be set in advance by a predetermined shape or size. The operator can set the region of interest having a predetermined shape or size (for example, a circle having about 5 mm diameter) in plaque 32 by clicking plaque 32 via console 18.

(S104)

Controller 17 outputs Position, Shape, Size, Number, Etc. of each region of interest 40 set by console 18 to elasticity information analyzing unit 22. Elasticity information analyzing unit 22 reads out the elasticity information for α-period (one heartbeat) from among the elasticity information for a period of several heartbeats in the plurality of regions of interest 40, and analyzes the elasticity information in α-period. Then the operator pushes measurement button 38 shown in FIG. 4(c) using console 18, and measurement of the respective regions of interest 40 is started.

Elasticity information analyzing unit 22 obtains the information on time variation of the strain in the respective regions of interest 40 from elasticity information calculating unit 13. In the graph of FIG. 4(b), the lateral axis indicates time, the longitudinal axis indicates strain, and each alphabet indicates regions of interest A˜E respectively. Here, strain in each region of interest 40 means the average value of the strain in the respective regions of interest. Elasticity information analyzing unit 22 analyzes the feature amount (for example, strain maximum value, strain minimum value, strain variation rate, etc.) from the information on time variation of the strain. In this time variation information of the strain, the largest strain maximum value is of region of interest A and the smallest strain maximum value is of region of interest B as shown in FIG. 4(b).

(S105)

Image display unit 10 displays time variation information and the feature amount (for example, strain maximum value, strain minimum value, strain variation rate, etc.) of the strain in the respective regions of interest 40 shown in FIG. 4(b).

(S106)

Hue setting unit 23 sets the hue of an elasticity image based on the feature amount of the elasticity information on the respective regions of interest analyzed by elasticity information analyzing unit 22. For example, hue setting unit 23 arranges the strain maximum values obtained from the respective regions of interest in descending order, and sets the hue of red, yellow, green, light green and blue in descending order of the strain maximum values. As shown in FIG. 4(b), the strain maximum values are a>c>e>d>b. Therefore, hue setting unit 23 sets red when the strain is a˜c, yellow when the strain is c˜e, green when the strain is e˜d, light green when the strain is d˜b and blue when the strain is b˜0 as shown in FIG. 4(a). While the hue is divided into five phases since five regions of interest 40 are set in this case, the hue phases, the range of strain in the respective hue phases or the hue can be set as desired by the operator using console 18.

(S107)

Hue Setting Unit 23 outputs the Set Hue Information to Color scan converter 15. By pushing image reconstruction button 39 in FIG. 4(c), color scan converter 15 appends the hue set by hue setting unit 23 to the elasticity image data from elasticity image constructing unit 14 and reconstructs the image. Color scan converter 15 converts the elasticity image data into red (R), green (G) and blue (B) with the set hue. Image display unit 10 displays the reconstructed elasticity image.

FIG. 6(a) is the elasticity image before being reconstructed, and FIG. 6(b) is the reconstructed elasticity image. Coloration in the vicinity of the respective regions of interest 40 is different in the reconstructed elasticity image. Even in plaque 32, different elasticity characteristics can be visually recognized.

As mentioned above, in accordance with the present embodiment, it is possible to set the display modes of an elasticity image according to the characteristics of biological tissues, whereby the hue in the same tissue can be clarified and the diseased area in the same tissue can be easily identified.

While a biological tissue is specified as a plaque of carotid artery 30 in the present embodiment, the present embodiment can be applied also to other biological tissues such as a tumor in mammary gland or prostate gland, or orthopedic field of limbs. Also, image display unit 10 is capable of zooming and displaying a tomographic image, an elasticity image, a biological tissue (plaque 32) in an elasticity image or a plurality of regions of interest.

### Second Embodiment

### Automatic

The second embodiment will be described here referring to FIG. 7˜FIG. 11. A difference from the first embodiment is that a plurality of regions of interest can be set automatically. Diagrams and description on operation procedure of the second embodiment (including the ultrasonic image display method and ultrasonic diagnostic program) are omitted here since it is the variation of procedure operation S103 in the first embodiment.

FIG. 7 is the device configuration of the second embodiment. In addition to the device configuration in the first embodiment, the second embodiment further comprises region of interest setting unit 24 configured to set a region of interest using the tomographic image data constructed in tomographic image data constructing unit 7.

The tomographic image data constructed in tomographic image constructing unit 7 are outputted to region of interest setting unit 24, and region of interest setting unit 24 analyzes luminance information of the tomographic image data. In concrete terms, first the outer frame of plaque 32 in the tomographic image is specified by console 18, and controller 17 outputs the specified information on the outer frame to region of interest setting unit 24.

Also, region of interest setting unit 24 may specify the outer frame of plaque 32 using the characteristics of plaque 32. The characteristics of plaque 32 are, for example, that it is attached to the surface of wall 31 in artery carotid 30 or that there are no Doppler signals that are from flowing blood.

In concrete terms, region of interest setting unit 24 acquires luminance distribution in the thickness direction of wall 31 in the tomographic image data. Then it sets the local maximum point having the maximum luminance in the acquired luminance distribution as the outer membrane reference point, and the second local maximum point which appears on the inner side (bloodstream side) from the outer membrane reference point as the inner membrane reference point. It recognizes the tissues having high luminance which is on the inner side (bloodstream side) from the inner membrane reference point. Further, it recognizes the region without Doppler signals as plaque 32 from among the recognized tissues having high luminance, and specifies the outer frame of plaque 32.

Region of interest setting unit 24 divides the luminance of the tomographic image in the specified plaque 32 into a plurality of phases (for example, 5 phases). For example, when the luminance has 256 gradations and the luminance of plaque 32 is in the range of 1˜150, region of interest setting unit 24 divides the luminance into 5 phases of, for example 1˜30, 31˜60, 61˜90, 91˜120 and 121˜150.

Then region of interest setting unit 24 sets five regions of interest as shown in FIG. 8(b) in accordance with the luminance distribution analyzed as shown in FIG. 8(a). Region of interest A′ falls in the range of luminance 1˜30, region of interest B′ falls in the range of luminance 31˜60, region of interest C′ falls in the range of luminance 61˜90, region of interest D′ falls in the range of luminance 91˜120, and region of interest E′ falls in the range of luminance 121˜450. In this manner, regions of interest A′˜E′ are set based on the luminance of the tomographic image data.

Also, when the luminance of plaque 32 falls in the range of 1˜90, region of interest setting unit 24 divides the luminance into five phases of 1˜30, 31˜45, 46˜60, 61˜75 and 76˜90. Region of interest A′ falls in the range of luminance 1˜30, region of interest B′ falls in the range of luminance 31˜45, region of interest C′ falls in the range of luminance 46˜60, region of interest D′ falls in the range of luminance 61˜75, and region of interest E′ falls in the range of luminance 76˜90. Since it is generally considered that the plaque having low luminance has high risk of breaking down, region of interest setting unit 24 sets region of interest A′ in the range of low luminance, for example the range of luminance 1˜30.

Then the position, shape, size, number etc. of the respective regions of interest 40 set by region of interest setting unit 24 are outputted to elasticity information analyzing unit 22. Elasticity information analyzing unit 22 reads out the elasticity information of α-period from among the elasticity information for a period of several heartbeats in the plurality of regions of interest 40 from elasticity information calculating unit 13, and analyzes the elasticity information in α-period.

Elasticity information analyzing unit 22 analyzes the feature amount (for example, strain maximum value, strain minimum value, strain variation rate, etc.) from time variation information of the strain in the respective regions of interest, as in the first embodiment. Then hue setting unit 23 sets the hue of the elasticity image based on the feature amount of the elasticity information in the respective regions of interest analyzed by elasticity information analyzing unit 22. Color scan converter 15 appends the hue set by the hue setting unit 23 to the elasticity image data from elasticity image constructing unit 14, and reinstructs the image. Color scan converter 15 then converts the elasticity image data into red (R), green(G) and blue(B) by the set hue. Image display unit 10 displays the reconstructed image.

As described above, in accordance with the present embodiment, it is possible to set display mode of elasticity images automatically according to the characteristics of biological tissues, whereby making it possible to clarify the hue in the same tissue and to identify a diseased area in the same tissue.

Also as shown in FIG. 9, regions of interest 40 may be newly set with respect to regions of interest A′˜E′ set by region of interest setting unit 24. Regions of interest 40 which are respectively smaller than regions of interest A′˜E′ are re-set in regions of interest A′˜E′ respectively by console 18 using the same method described in S103 of the first embodiment. Also while region of interest setting unit 24 sets regions of interest 40 in accordance with the luminance of a tomographic image above, regions of interest can be also set using the methods shown in FIG. 10 and FIG. 11.

(Scanning Direction Line)

FIG. 10 shows the method which divides plaque 32 at predetermined scanning direction line intervals 41 to set a plurality of regions of interest 40. Region of interest setting unit 24 sets a plurality of scanning direction lines 41 and divides a tomographic image into a plurality of regions. For example, six scanning direction lines 41 are displayed at 5 mm intervals, and the tomographic image is divided into five regions.

Then the outer frame of plaque 32 in the tomographic image is specified by console 18, and controller 17 outputs the specified outer frame information to region of interest setting unit 24. Also, region of interest setting unit 24 may specify the outer frame of plaque 32 using the characteristics of plaque 32.

Region of interest setting unit 24 sets the region wedged between six scanning direction lines 41 and the outer frame of plaque 32 set in the tomographic image as region of interest 40. Here, region of interest setting unit 40 sets the region on the far left as region of interest A, and the region on the right of region of interest A as region of interest B. Then it sets the region on the right of region of interest B as region of interest C, the region on the right of region of interest C as region of interest D, and the region on the right of region on the far right as region of interest E. Region of interest setting unit 24 does not set region of interest 40 in the case that the region wedged between two scanning direction lines 41 and plaque 32 set in an tomographic image is dimensionless (for example, smaller than 1 mm²).

(Lattice)

FIG. 10(b) shows the method that divides plaque 32 in a reticular pattern by scanning direction lines 42 and scanning direction lines 43 that are vertically intersecting with scanning direction lines 42 so as to set a plurality of regions of interest 40. Region of interest setting unit 24 sets a plurality of scanning direction lines 42 and scanning direction lines 43, and divides a tomographic image into a plurality of regions. For example, six scanning lines 41 are displayed at 5 mm intervals and three scanning direction lines 43 are displayed at 2 mm intervals, and the tomographic image is divided into ten regions.

Then the operator specifies the outer frame of plaque 32 on the tomographic image by console 18, and controller 17 outputs the specified outer frame information to region of interest setting unit 24. Also, region of interest setting unit may specify the outer frame of plaque 32 using the characteristics of plaque 32.

Region of interest 24 sets the region wedged between six scanning direction lines 42, three scanning direction lines 43 and the outer frame of plaque 32 set on the tomographic image as region on interest 40. Here, region of interest setting unit 24 sets the region on the upper left end as region of interest A, and the region on the right of region of interest A as region of interest B. Then it sets the region on the right of region of interest B as region of interest C, and the region on the right of region of interest C as region of interest D. In the case that the region wedged between scanning direction lines 42, scanning direction lines 43 and the outer frame of plaque 32 is dimensionless (for example, smaller than 1 mm²), for example the case of the region on the right of region of interest D, region of interest setting unit 24 does not set region of interest 40. The lower stage of region of interests 40 are set in the same manner as the upper stage of regions of interest 40.

(Plaque Surface)

FIG. 11 shows the method that sets a plurality of regions of interest 40 on the surface of plaque 32. The operator specifies the outer frame of plaque 32 on a tomographic image by console 18, and controller 17 outputs the specified outer frame information to region of interest setting unit 24. Also, region of interest setting unit 24 may specify the outer frame of plaque 32 using the characteristics of plaque 32.

Region of interest setting unit 24 sets a rectangle region of interest 40 along the specified outer frame of plaque 32. Region of interest setting unit 24 analyzes the border between the portion without blood flow signals and the region with blood flow signals as the surface of plaque 32 using, for example a Doppler signal, and sets the region of interest 40 on the border thereof. Therefore, region of interest 40 is not set between plaque 32 without blood flow signal and wall 31 without blood flow signal. In other words, region of interest 40 is set only on the surface of plaque 32. Also, region of interest setting unit 24 sets region of interest 40 in the way that the normal direction of the outer frame of plaque 32 and the longitudinal direction of region of interest 40 are approximately correspondent.

Since regions of interest A˜F are disposed along the surface of plaque 32 and an elasticity image is reconstructed by the hue being set based on the feature amount of elasticity information in the respective regions of interest 40, the risk that plaque 32 breaks down can be recognized.

### Third Embodiment

### Color Tomographic Image

The third embodiment will be described here referring to FIG. 3 and FIG. 7.

The difference from the first and the second embodiments is that a color tomographic image is displayed.

As shown in FIG. 7, the third embodiment comprises color tomographic image constructing unit 25 configured to construct a color tomographic image using the tomographic image data constructed in tomographic image constructing unit 7. Tomographic image constructing unit 25 sets, for example red for region of interest region A′ in the range of luminance 1˜30, yellow for region of interest B′ in the range of luminance 31˜60, green for region of interest C′ in the range of luminance 61˜90, light green for region of interest D′ in the range of luminance 91˜120 and blue for region of interest E′ in the range of luminance 121˜.

Image display unit 10 displays plaque 32 of the tomographic image shown in FIG. 3(a) on the color tomographic image constructed in color tomographic image constructing unit 25. When the luminance on the tomographic image is, for example less than 30, since plaque 32 has tendency of breaking down (a part of plaque 32 tends to fall off), the risk of plaque 32 breaking down can be identified by the operator by paying attention on the red region.

Also while a tomographic image is displayed independently above as shown in FIG. 3(a), a synthetic image in which the reconstructed tomographic image and a color tomographic image are superimposed may also be displayed as shown in FIG. 3(b).

In concrete terms, switching and adding unit 9 adds in each coordinate red (R) value, green (G) value and blue (B) value of a tomographic image and red (R) value, green (G) value and blue (B) value of a color tomographic image respectively as shown in equation 1 below.

$$( Synthetic image data R ) = \frac{1}{2} \times ( Elasticity image data R ) + \frac{1}{2} \times ( Color tomographic image data R ) , \\ ( Synthetic image data G ) = \frac{1}{2} \times ( Elasticity image data G ) + \frac{1}{2} \times ( Color tomographic image data G ) , \\ ( Synthetic image data B ) = \frac{1}{2} \times ( Elasticity image data B ) + \frac{1}{2} \times ( Color tomographic image data B ) \qquad [ Equation 1 ]$$

Then the synthetic image in which an elasticity image and a color tomographic image are superimposed that is created in switching and adding unit 9 is to be displayed in FIG. 3(b). While the adding ratio is set as ½ in the present embodiment, switching and adding unit 9 may also set the adding ratio in the range of 1˜0. The operator can identify the risk of plaque 32 breaking down by paying attention to red color in synthetic image data for identifying the region where it is soft and has low luminance.

In the same manner, a tomographic image and a color tomographic image also can be superimposed and displayed on FIG. 3(a). In concrete terms, switching and adding unit 9 adds in each coordinate red (R) value, green (G) value and blue (B) value of a tomographic image and red(R) value, green (G) value and blue (B) value of a color tomographic image respectively as shown in equation 2 below.

$$( Synthetic image data R ) = \frac{1}{2} \times ( Tomographic image data R ) + \frac{1}{2} \times ( Color tomographic image data R ) , \\ ( Synthetic image data G ) = \frac{1}{2} \times ( Tomographic image data G ) + \frac{1}{2} \times ( Color tomographic image data G ) , \\ ( Synthetic image data B ) = \frac{1}{2} \times ( Tomographic image data B ) + \frac{1}{2} \times ( Color tomographic image data B ) \qquad [ Equation 2 ]$$

Then the synthetic image in which a tomographic image and a color tomographic image that is created in switching and adding unit 9 is to be displayed in FIG. 3(a).

As described above, in accordance with the present embodiment, a display mode of a tomographic image can be set according to the characteristics of biological tissues, whereby making it possible to identify a diseased area in the same tissue.

(Alarm)

A fourth embodiment will be described here referring to FIG. 1 and FIG. 7. A difference from the first˜third embodiments is that when there is a high-risk region in biological tissues, the high-risk region is enhanced in display.

In the case that an alarm is to be displayed on a color tomographic image, color tomographic image constructing unit 25 causes the color tomographic image of region of interest A′ which is in the range of luminance 1˜30 to blink. When the luminance of the tomographic image is less than 30, it means that plaque 32 is at high risk of breaking down, thus the operator can pay attention on the high-risk region by blinking of the tomographic image of the region thereof.

In the case of displaying an alarm on region of interest 40, the regional frame of region of interest A′ being set by region of interest setting unit 24 can also be enhanced in display.

Also, in the case of displaying an alarm on a tomographic image, the region of interest having high strain is to be made to blink. In time variation information of the strain shown in FIG. 4(b), the strain maximum value of region of interest A is the maximum and the strain maximum value of region of interest B is the minimum. An elasticity image is to be made to blink based on the feature amount of the elasticity information on the respective regions of interest analyzed by elasticity information analyzing unit 22.

While an image is enhanced in display by blinking it in the above description, the image to be enhanced may be displayed by another methods besides blinking such as flashing or using arrows.

As described above, in accordance with the present embodiment, a display mode of a tomographic image or an elasticity image may be set according to the characteristics of biological tissues, whereby making it possible to identify a diseased area in the same tissue.

(Timing)

A fifth embodiment will be described using FIG. 1, FIG. 3 and FIG. 7.

While a region of interest is set by freezing a tomographic image or elasticity image in the first˜fourth embodiments, the timing for setting a region of interest or for analyzing feature amount of elasticity information is to be controlled in the present embodiment.

Though not shown in the diagram, an R-wave time phase detecting unit configured to detect the R-wave time phase to be the basis from among the obtained electrocardiographic waveforms and an R-wave delay pulse generating unit configured to generate a timing pulse capable of having a desired time phase set by an operator by inputting it via console 18 on the basis of the R-wave time phase are comprised. Image display unit 10 freezes the tomographic image or elasticity image at the time phase which is delayed from the R-wave time phase acquired by the R-wave delay pulse generating unit. The time phase which is delayed from the R-wave time phase is in the condition that pressure is applied on the entire carotid artery 30, thus the elasticity image is properly displayed. Therefore, a region of interest can be appropriately set by setting it using the elasticity image at this time phase as in the same manner in the first embodiment.

Also, the time phase which is delayed from the R-wave time phase is in the condition that pressure is applied on the entire carotid artery 30, elasticity information analyzing unit 22 may read out from elasticity information calculating unit 13 the feature amount (strain and elasticity modulus) of elasticity information at the time phase (which is delayed from the R-wave time phase) from among the elasticity information for a period of several heartbeats in a plurality of regions of interest 40 and analyze the elasticity information. Then hue setting unit 23 sets the hue of the elasticity information based on the feature amount of elasticity information in the respective regions of interest analyzed by elasticity information analyzing unit 22 as in the same manner in the first embodiment.

While electrocardiographic waveforms are used as describing the present embodiment, pressure information of pressure measuring unit 16 may also be used.

(Operation Procedure)

Operation procedure in the case that the respective embodiments of the present invention are combined will be described referring to FIG. 12.

(S201) First, tomographic image constructing unit 7 and elasticity image constructing unit 14 construct tomographic image data and elasticity image data of biological tissues (here plaque 32 of carotid artery 30), and displays them on image display unit 10.

(S202) The operator freezes the tomographic image and the elasticity image using console 18. Elasticity information calculating unit 13 stores the calculated elasticity information for a period of several heartbeats before the information is frozen.

(S203) The operator selects the region of interest setting mode choosing whether to set region of interest 40 manually or automatically via console 18.

(S204) In the case of setting region of interest 40 manually, it is to be set using the method in the above-described embodiment 1 via console 18.

(S205) In the case of setting region of interest 40 automatically, it is to be set by region of interest setting unit 24 using the method in the above-described second embodiment.

(S206) The operator selects whether or not to display a color tomographic image using color tomographic image constructing unit 25 via console 18. In the case of not displaying a color tomographic image, S210 is to be proceeded.

(S207) Color tomographic image constructing unit 25 constructs a color tomographic image using the method in the above-described third embodiment, and displays it on image display unit 10.

(S208) The operator selects whether or not to re-set region of interest 40 via console 18. In the case of not re-setting region of interest 40, 5210 is to be proceeded.

(S209) The operator re-sets region of interest 40 via console 18 using the method in the above-described first embodiment, then proceeds to S210.

(S210) Controller 17 outputs the position, shape, size, number etc. of the respective regions of interest 40 set in S204, S205 and S209 to elasticity information analyzing unit 22. Elasticity image information analyzing unit 22 reads out from elasticity information calculating unit 13 the elasticity information for α-period (one heartbeat) from among the elasticity information for a period of several heartbeats in a plurality of regions of interest 40, and analyzes the elasticity information in α-period. Elasticity information analyzing unit 22 analyzes the feature amount from time variation information of the strain.

(S211) Hue setting unit 23 sets the hue of elasticity information based on the feature amount of elasticity information in the respective regions of interest analyzed by elasticity information analyzing unit 22.

(S212) Hue setting unit 23 outputs the set hue information to color scan converter 15. Color scan converter 15 appends the hue set by hue setting unit 23 to the elasticity image data from elasticity image constructing unit 14, and reconstructs the image. Image display unit 10 displays the reconstructed elasticity image.

As described above, since the operator can select the steps of the operation as desired, it is possible to make diagnosis which is suited for a patient or an examining region.

### DESCRIPTION ON REFERENCE NUMERALS

1: object, 2: ultrasonic probe, 3: transmission unit, 4: reception unit, 5: ultrasonic transmission/reception controller, 6: phasing and adding unit, 7: tomographic image constructing unit, 8: black and white scan converter, 9: switching and adding unit, 10: image display unit, 11: RF signal frame data selecting unit, 12: displacement calculating unit, 13: elasticity information calculating unit, 14: elasticity image constructing unit, 15: color scan converter, 16: pressure measuring unit, 17: controller, 18: console, 22: elasticity information analyzing unit, 23: hue setting unit, 24: region of interest setting unit, 25: color tomographic image constructing unit

1. An ultrasonic diagnostic apparatus comprising:
an ultrasonic probe configured to transmit/receive ultrasonic waves to/from an object to be examined;
a transmission unit configured to transmit ultrasonic waves via the ultrasonic probe;
a reception unit configured to receive reflected echo signals from the object:
an elasticity information calculating unit configured to calculate strain or elasticity modulus by the RF signal frame data based on the reflected echo signals received by the reception unit;
an elasticity image constructing unit configured to construct an elasticity image based on the strain or elasticity modulus obtained by the elasticity information calculating unit;
a tomographic image constructing unit configured to construct a tomographic image based on the RF signal frame data; and
an image display unit configured to display the tomographic image and/or the elasticity image,
characterized in further comprising:
an elasticity information analyzing unit configured to set a plurality of regions of interest on the tomographic image or the elasticity image and analyze the feature amount of elasticity information in the plurality of regions of interest; and
a hue setting unit configured to set the hue of the elasticity image based on the feature amount.

2. The ultrasonic diagnostic apparatus according to claim 1, wherein the elasticity information analyzing unit reads out elasticity information of the plurality of regions of interests at a predetermined time from the elasticity information calculating unit and analyzes the feature amount of the elasticity information.

3. The ultrasonic diagnostic apparatus according to claim 1, wherein the feature amount is obtained from time variation information of a strain.

4. The ultrasonic diagnostic apparatus according to claim 3, wherein the time variation information of a strain is one of a strain maximum value, strain minimum value or strain variation rate.

5. The ultrasonic diagnostic apparatus according to claim 1, wherein the hue setting unit allots the hue based on the size of the feature amount of the elasticity information obtained from the plurality of regions of interest.

6. The ultrasonic diagnostic apparatus according to claim 1, wherein the region of interest is set by a predetermined shape or size in advance, characterized in further comprising a console configured to set the plurality of regions of interest.

7. The ultrasonic diagnostic apparatus according to claim 1, characterized in further comprising a region of interest setting unit configured to set the plurality of regions of interest in accordance with luminance distribution of the tomographic image.

8. The ultrasonic diagnostic apparatus according to claim 1, characterized in further comprising a region of interest setting unit configured to set a plurality of lines on the tomographic image or elasticity image and set the regions segmentized by the plurality of lines as the plurality of regions of interest.

9. The ultrasonic diagnostic apparatus according to claim 1, characterized in further comprising a region of interest setting unit configured to set the plurality of regions of interest along the outer frame of an examination target region of the object.

10. The ultrasonic diagnostic apparatus according to claim 1, characterized in further comprising a color tomographic image constructing unit configured to construct a color tomographic image based on luminance of the tomographic image.

11. The ultrasonic diagnostic apparatus according to claim 10, wherein the color tomographic image constructing unit, in the case that luminance of the tomographic image is less than a predetermined value, highlights the tomographic image having less luminance than the predetermined value.

12. The ultrasonic diagnostic apparatus according to claim 1, wherein the elasticity information analyzing unit reads out feature amount of the elasticity information corresponding to the electrocardiographic time phase from among the elasticity information at a predetermined time in the plurality of regions of interest and analyzes feature amount of the elasticity information.

13. The ultrasonic diagnostic apparatus according to claim 1, wherein the image display unit executes zoom-display of an elasticity image, tomographic image and biological tissues on an elasticity image.

14. An ultrasonic image display method including:
a step that constructs an elasticity image based on strain or elasticity modulus by ultrasonic signals;
a step that constructs a tomographic image by ultrasonic signals;
a step that sets a plurality of regions of interest on the tomographic image or the elasticity image;
a step that analyzes feature amount of elasticity information in a plurality of regions of interest;
a step that sets a hue of the elasticity image based on the feature amount; and
a step that displays the elasticity image based on the set hue.

15. An ultrasonic diagnostic program including:
a function to construct an elasticity image based on strain or elasticity modulus by ultrasonic signals;
a function to construct a tomographic image by ultrasonic signals;
a function to set a plurality of regions of interest on the tomographic image or the elasticity image;
a function to analyze feature amount of elasticity information in a plurality of regions of interest;
a function to set a hue of the elasticity image based on the feature amount; and
a function to display the elasticity image based on the set hue.
